(19) United States
(12) Patent Application Publication
(10) Pub. No.: US 2012/0226180 A1
(43) Pub. Date: Sep. 6, 2012

(54) USER UNIT FOR DETERMINING OUTPUT PARAMETERS FROM BREATH GAS ANALYSES

(75) Inventors: Michael Volkel, Hullern (DE); Dirk Tuchtenhagen, Furth (DE)

(73) Assignee: ACEOS GMBH, Furth (DE)

(21) Appl. No.: 13/390,229

(22) PCT Filed: Aug. 5, 2010
(86) PCT No.: PCT/EP2010/004803

§ 371 Date: May 1, 2012

(30) Foreign Application Priority Data
Aug. 11, 2009 (DE) 10 2009 036 901.5
Oct. 7, 2009 (DE) 10 2009 048 603.8
Jan. 13, 2010 (DE) 10 2010 004 611.6

(51) Int. Cl. A61B 5/097

(52) U.S. Cl. 600/532

(57) ABSTRACT

Shown and described is a user unit (1) for determining output parameters from respiratory gas analyses, particularly for use in spiroergometry devices, with a preferably replaceable breathing tube (2) and a housing part (3), wherein the breathing tube (2), also preferably, can be inserted into the housing part (3) in order to put the user unit into a measuring mode. A provision is made according to the invention that a nose clip (4) connected to the housing part (3) and designed to seal the nose of a user (5) during the determination of the output parameters is provided on the top side of the housing part (3).

The invention relates to a user unit for determining output parameters from respiratory gas analyses, particularly for use in spiroergometry devices, with a preferably replaceable breathing tube and a housing part, wherein the breathing tube, also preferably, can be inserted into the housing part in order to put the user unit into a measuring mode.

A precise and direct method for determining metabolic activity in humans is the analysis of the respiratory gases, in which the concentration of oxygen and carbon dioxide in the breath as well as the volume flow of the breath are determined. Metabolic values such as, for example, the respiratory quotient, also known as RQ, can be calculated on the basis of the measurements. RQ is the ratio of the amount of exhaled carbon dioxide to the amount of oxygen taken in. To determine these gas quantities, various parameters are measured. The flow volume of the respiratory gas is determined using a sensor which measures the flow velocity of the respiratory gas by measuring the ultrasound travel time, for example. Various respiratory volumes can be derived through the integration of the volume flow over time. In known spiroergometry devices, a gas sample is also suctioned off from the main respiratory flow and fed to the sensor system contained in the device. As a result, the concentrations of oxygen and carbon dioxide upon inhaling and exhaling can be determined. The respective values of the gas concentrations differ substantially between inhaling and exhaling. Using the previously determined respiratory volume, the gas quantities that were converted by the body can be calculated from the concentrations.

Conventional spiroergometry devices consist of a user unit and an analysis unit, the analysis unit containing the sensor system required for determining the gas concentrations. The subject interface is usually a user unit with a breathing mask on which a flow sensor has additionally been mounted which measures the respiratory flow. A gas sample is continuously suctioned off by means of a thin tube on the breathing mask and fed to the analysis unit, where the corresponding gas concentrations are measured. User units with breathing mask are used particularly for continuous measurements over a longer period of time.

To determine output parameters from respiratory gas analyses, it is necessary to measure the entire respiratory flow. In normal cases, the respiratory flow is divided upon exhaling into a nasal respiration portion and an oral respiration portion, and both portions must be detected in order to determine the output parameters. To do this, known breathing masks cover both mouth and nose of a subject. Consequently, the entire respiratory flow is fed through the mask. To prevent leaks, the mask must lie tightly against the subject's face. Secure attachment on the subject's head is achieved through elastic bands. However, the tight-fitting breathing mask can be quite unpleasant for the subject. In addition, it is associated with hygienic disadvantages, since it is contaminated by the breath of the subject during each use. If several people are to be tested one after the other, then the breathing mask must be disinfected after each use, which is laborious. What is more, infections may occur as a result of insufficient disinfection.

For so-called spot measurements, in which the output parameters from respiratory gases are only measured at the respective measuring points, so-called nose clips can be used to suppress the respiratory flow that usually flows through the nose. Nose clips can also be used together with such user units which have a preferably replaceable breathing tube and a housing part, the breathing tube being inserted into the housing part. Also preferably, the breathing tube can be disposable. Such a user unit is known, for example, from DE 42 22 286 C1.

A disposable nose clip is known from WO 2009/056562 A1 which can be fabricated using conventional methods such as milling, cutting or punching. The known nose clip has a substantially horseshoe or U shape. Two projecting arms extend from a base in substantially the same direction. Located between these two projecting arms of the nose clip is an open area which is designed for the nose to fit in it.

From DE 34 16 146 A1, another nose clip is known having two legs held together by a spring, the legs being made of a light material and the spring being just strong enough that it is able to press together the nasal walls to seal the nostrils. Using an adjustment screw, the pressure of the springs can be altered individually.

The known nose clips are superior to breathing masks by virtue of a greater level of comfort. The use of the known disposable nose clips enables the determination of output parameters from respiratory gas analyses while ensuring the necessary hygiene. Nonetheless, user errors can occur upon placement of the known nose clips onto the nose, as a result of which the nose may not be completely sealed. Nasal respiration is then not completely ruled out when the nose clip is in place, so a distortion of the measurement results may occur as a result of an air flow or respiratory flow through the nose.

It is the object of the present invention to provide a user unit of the type mentioned at the outset which is easy to handle for a test subject and is characterized during the determination of the output parameters by a high degree of comfort and measurement precision. Moreover, the user unit according to the invention can be produced at low cost and meets high hygienic standards.

The aforementioned objects are achieved by a user unit of the type mentioned at the outset in a first embodiment of the invention through the provision of a nose clip connected to the housing part on the top side of the housing part for sealing the nose during the determination of the output parameters. The invention is based on the basic idea of joining the nose clip to the housing part in such a way that user errors upon placing the nose clip onto the nose can be ruled out. The placement of the nose clip and the insertion of the mouthpiece can be done with a single movement of the hand, which constitutes a great relief for the test subject particularly during physical exertion on a treadmill, for example. As a result of the position of the user unit during the determination of the output parameters, i.e., the arrangement of the housing part and the breathing tube relative to the test subject, the position of the nose clip relative to the test subject is also established. The user unit according to the invention is pleasant to handle and enables the output parameters to be determined with great precision, and a distortion of the measurement results due to respiratory flow traveling through the nose can be ruled out to a large extent. In addition, the parts of the nose clip coming into contact with the test subject's nose can be disposable in order to meet strict hygienic requirements.

The user unit according to the invention has a breathing tube which is preferably disposable and a housing part into which the breathing tube can be introduced or inserted. At least one sampling point can be provided on the breathing tube and/or on the housing part for the removal of a gas sample from the main respiratory flow. The removed gas sample can then be fed through a tube to a separate analysis unit. The analysis of the respiratory gases can then be performed in the analysis unit. In principle, however, it is also possible for the user unit to have sensors for analyzing the respiratory gases. In addition, sensors, particularly ultrasonic transducers, can be provided on the housing part with which it is possible to calculate the velocity of the respiratory flow, the volume flow and various lung volumes. The gas concentrations in the respiratory gas sample, the velocity of the respiratory flow and various lung volumes can be among the output parameters that are to be determined using the user unit according to the invention, though this is not an exhaustive list.

To achieve the abovementioned objects, a provision is made in a user unit of the type mentioned at the outset in a second alternative embodiment of the invention that a chin rest connected to the housing part designed to rest against the chin of a user during the determination of the output parameters is provided on the lower side of the housing part. By virtue of the chin rest, the user unit is supported against the chin of the test subject during the determination of the output parameters, so that little effort is required in order to hold the user unit. This contributes to the easy handling of the user unit according to the invention. Moreover, by appropriately shaping the chin rest, a desired position of the user unit relative to the face of the test subject can be supported. This is advantageous particularly if the housing has a nose clip connected to the housing part on the top side such that the housing part is held by the nose clip on the test subject's nose after the insertion of the breathing tube into the test subject's mouth and is supported on the test subject's chin by the chin rest. The chin rest can aid in the exact orientation of the user unit and nose clip with respect to the test subject's face and nose and in ensuring that the test subject's nose is always completely sealed by the nose clip. Consequently, nasal respiration is ruled out and a high level of precision in the determination of the output parameters is ensured.

Although the nose clip and the chin rest are preferably attached to the housing part, with the housing part being designed and intended for multiple use, it is possible in principle for the nose clip and/or the chin rest to be attached to the breathing tube and also be intended for one-time use. This aspect has inherent inventive significance.

The aforementioned aspects and features of the present invention as well as the aspects and features described below can be implemented independently of each other or in any combination. Further advantages, features, characteristics and aspects of the present invention follow from the following description of a preferred embodiment with reference to the drawing.

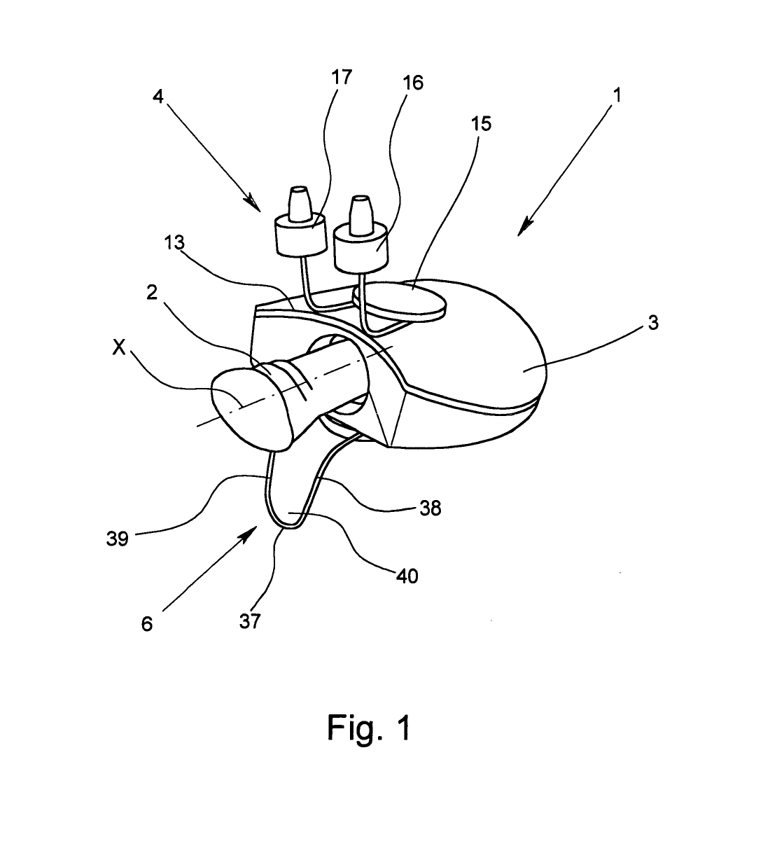

FIG. 1 shows a perspective view of a user unit according to the invention at an angle from above,

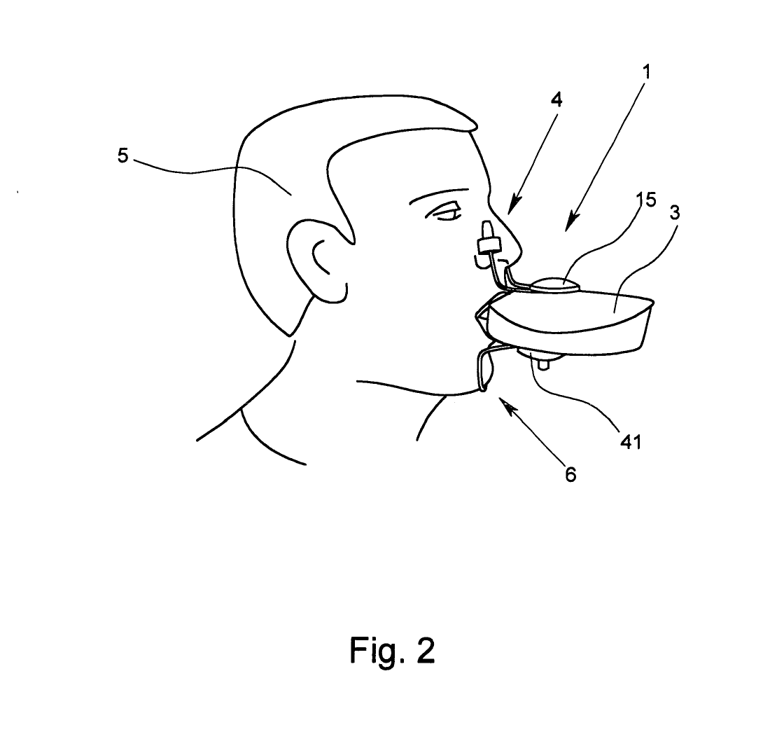

FIG. 2 shows a schematic representation of the user unit depicted in FIG. 1 during the determination of a test subject's output parameters,

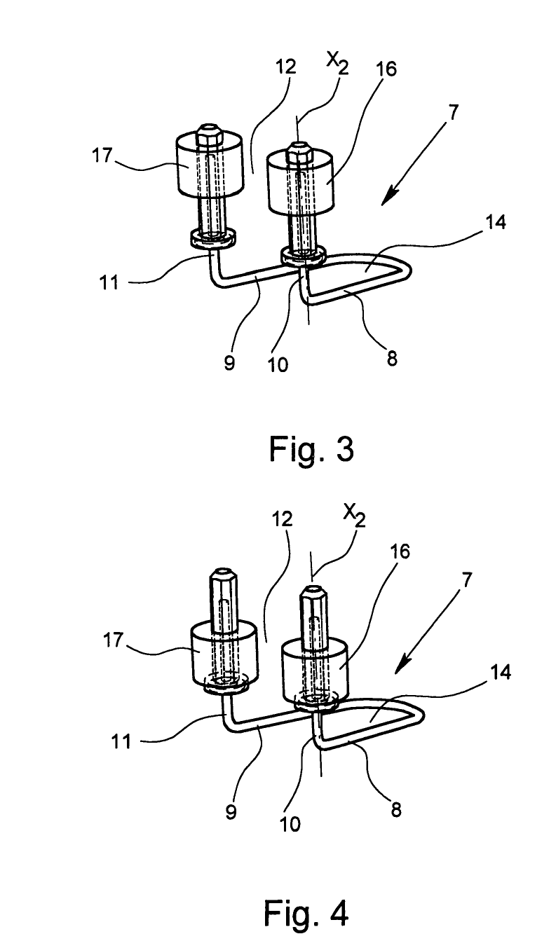

FIG. 3 shows a perspective view of a nose clip of the user unit depicted in FIG. 1 with clamp rollers moved upward,

FIG. 4 shows the nose clip depicted in FIG. 3 with clamp rollers moved downward,

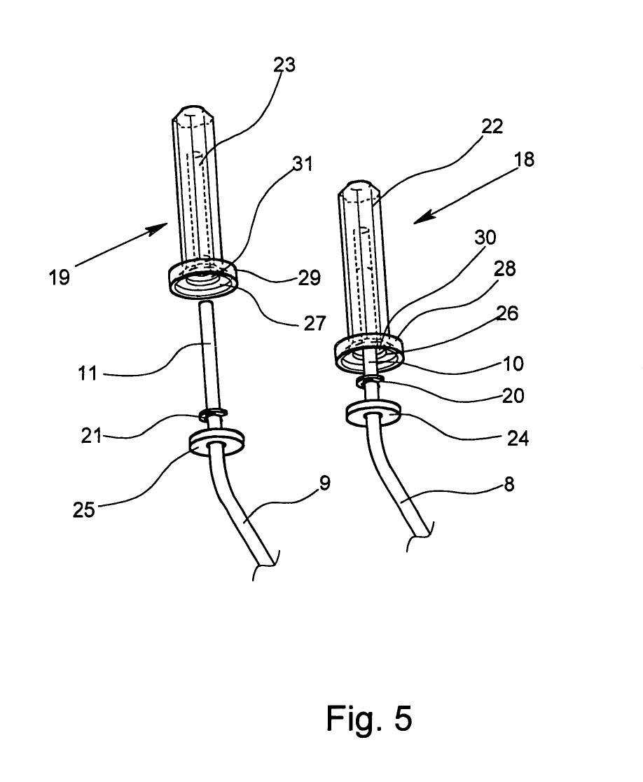

FIG. 5 shows a partial view of a clamping bracket of the nose clip depicted in FIGS. 3 and 4 with two roller bodies and with the roller bodies in a partially assembled state,

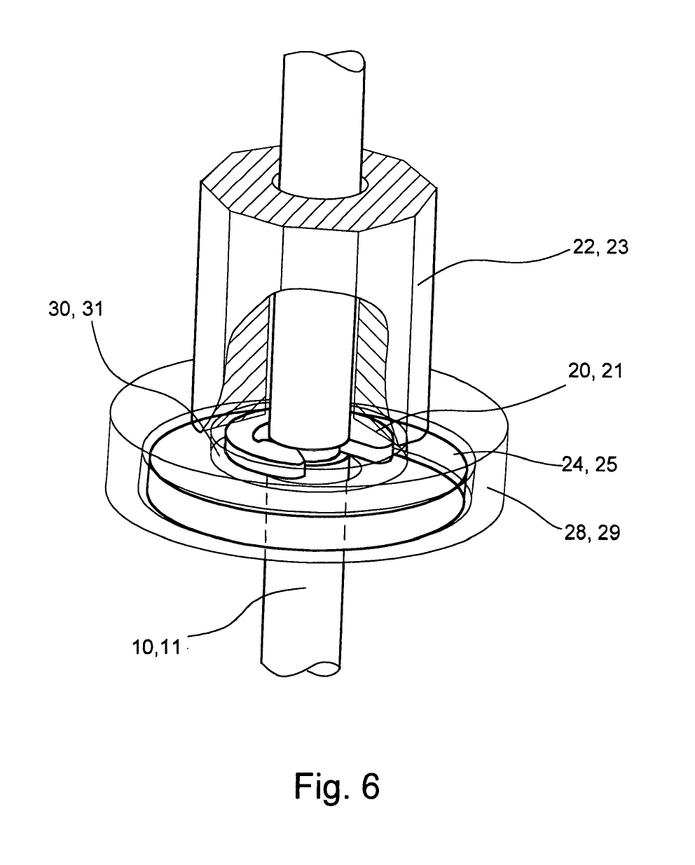

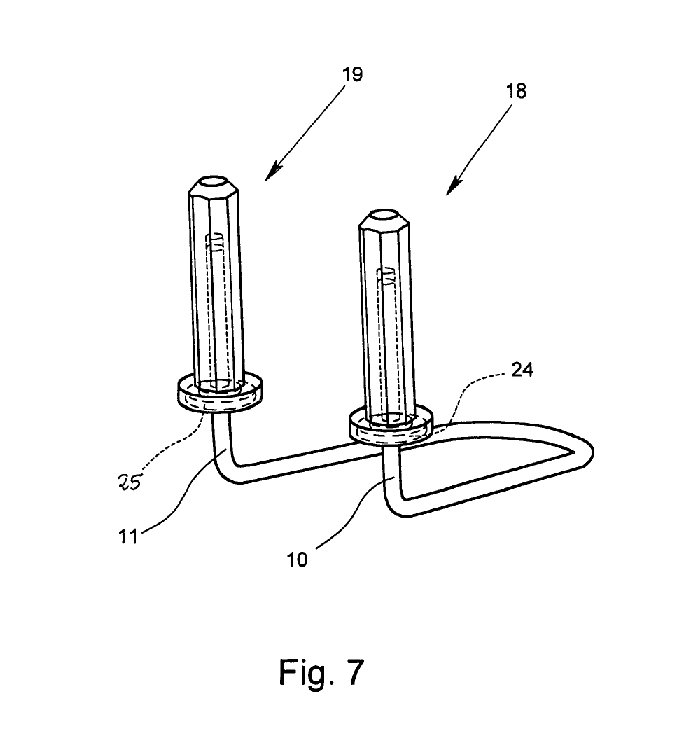

FIG. 6 shows a partial view of a roller body from FIG. 5 in the assembled state,

FIG. 7 shows a partial view of the clamping bracket depicted in FIG. 5 with the roller bodies in the assembled state,

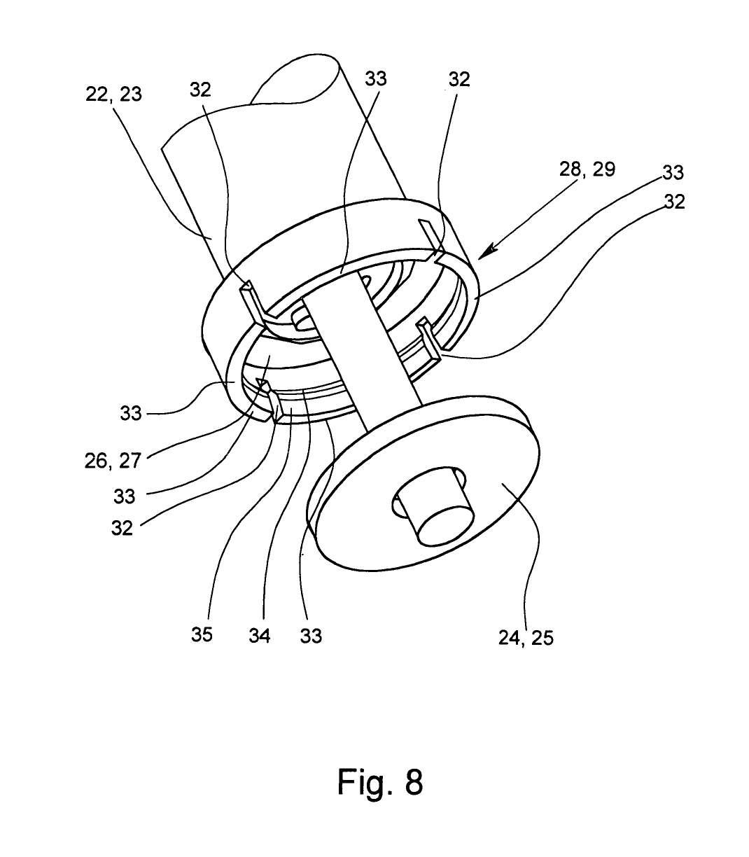

FIG. 8 shows a partial view of a clamping arm of the nose clip depicted in FIGS. 3 and 4 with another roller body, the roller body being in a disassembled state, and

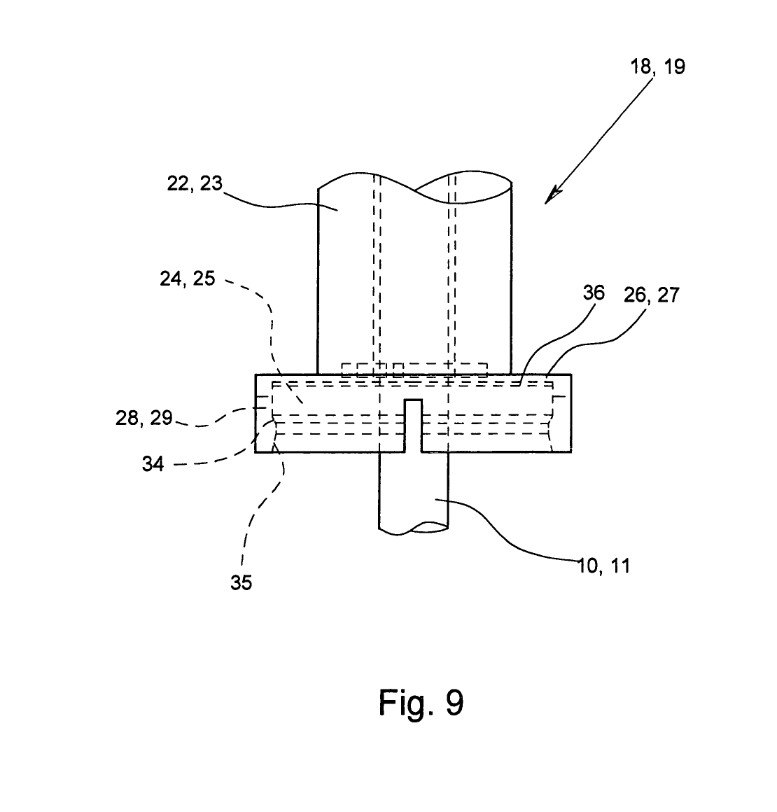

FIG. 9 shows the clamping arm depicted in FIG. 8 with the roller body, the roller body being in an assembled state.

FIGS. 1 and 2 show a schematic representation of a user unit 1 for use during the determination of the output parameters from respiratory gas analyses, particularly for use in spiroergometry devices. The user unit 1 has a preferably replaceable breathing tube 2 and a housing part 3, the breathing tube 2 being optionally inserted or pushed into the housing part 3 in order to put the user unit 1 in a ready-to-use state or measuring mode.

A nose clip 4 connected to the housing part 3 is provided on the top side of the housing part 3. The nose clip 4 serves to seal off the nose of a user 5 during the determination of the output parameters. As also follows from FIGS. 1 and 2, a chin rest 6 connected to the housing part 3 can be embodied on the bottom side of the housing part 3 to rest against the chin of the user 5 during the determination of the output parameters. The user unit 1 depicted in FIGS. 1 and 2 can be handled in a simple manner by a user 5 during the determination of the output parameters. In particular, it is not necessary for the user to hold the housing part 3 with his or her hands during the determination of the output parameters. The housing part 3 is held onto the nose by means of the breathing tube 2 using the mouth and additionally by means of the nose clip and is supported on the chin of the user 5 by means of the chin rest 6. The nose clip 4 ensures that nasal respiration is ruled out during the determination of the output parameters. As a result, the user unit 1 contributes to a high degree of precision in the determination of the output parameters.

Incidentally, one thing that is not shown is that the user unit 1 can be connected to an analysis unit via a hose connection, which analysis unit can contain a suctioning device and the sensor system necessary for the determination of the gas concentrations in the respiratory gas. Moreover, measuring devices such as ultrasonic transducers can be provided in the housing part 3 in order to determine, in a manner inherently known from the prior art, the volume flow and/or lung volumes upon inhaling and/or upon exhaling.

Also not shown is that the user unit 1, in an alternative embodiment, can also merely have a nose clip 4 connected to the housing part 3 or a chin rest 6 connected to the housing part 3.

The nose clip 4 can be mounted on the housing part 3 in the longitudinal direction X₁ of the breathing tube 2 in a depth-adjustable manner and have the option of being fixable at various points on the housing part 3. As a result, the spacing of the nose clip 4 from the face of the user 5 can be changed and the position of the nose clip 4 relative to the position of the nose of the user 5 can be preset. What is more, a provision can be made that the nose clip 4, in addition to the depth-adjustability, can also be moved crossways to the longitudinal direction X₁ of the breathing tube 2 or in the radial direction in order to adapt to the shape of the nose. This enables the complete sealing of the nose during the measurement of the output parameters.

As follows from FIGS. 3 through 9, the nose clip 4 can have a substantially U-shaped clamping bracket 7 with two clamping arms 8, 9, the two clamping arms 8, 9 being optionally angled in an L-shape at the free ends 10, 11. A clamping area 12 which is open toward the side of a mouthpiece of the breathing tube 2 or toward the side of the user 5 for the insertion of the nose of the user 5 is then formed between the angled ends 10, 11. A bent steel wire, for example a stainless steel wire, can be provided as a clamping bracket 7 with a diameter of 1 mm to 5 mm, preferably approximately 2 mm. This ensures sufficient clamping force of the clamping arms 8, 9.

As is clear from FIGS. 1 and 2, the free ends 10, 11 of the clamping arms 8, 9 can be arranged at an angle of less than or equal to 90° to the middle longitudinal axis X₁ of the breathing tube 2. The free ends 10, 11 of the clamp arms 8, 9 then extend forward beyond a front-side outer edge 13 of the housing part 3, enabling easy insertion of the nose of the user 5 into the clamping area 12. The housing part 3 can also have a concave arch on the front side facing the user 5 which makes it possible to guide the face of the user 5 closer to the nose clip 4.

To enable the easy depth-adjustment of the nose clip 4, the clamping bracket 7 can be fixed to the housing part 3 by means of a screw joint with at least one clamping screw, preferably in a U-shaped connection area 14 of the two clamping arms 8, 9. The U-shaped connection area 14 is shown in FIGS. 3 and 4. The clamping screw is screwed into the housing part 3 in the area between the two clamping arms 8, 9, the clamping bracket 7 being tensioned between a screw head 15 of the clamping screw and the housing part 3. In this way, the clamping bracket 7 can be moved more or less forward or backward in the longitudinal direction of the breathing tube 2 prior to the tightening of the clamping screw, in each case with reference to the face of the user 5. Through the tensioning of the clamping screw 7 between the screw head 15 and the housing part 3, the clamping bracket 7 is held onto the housing part 3 such that it cannot be lost, and it is fixed in place with the desired spacing from the nose of the user 5 by tightening the clamping screw. Through the optimal spacing of the free ends 10, 11 of the nose clip 4 from the nose of the user 5, a comfortable fit of the nose clip 4 is made possible, so that the clamping effect is not perceived by the user 5 as being bothersome. Moreover, as a result of the optimal spacing of the nose clip 4 from the face of the user 5, a maximally complete sealing of the nose is ensured during the determination of the output parameters.

It is not shown in detail that at least one guide projection and/or guide slot can be provided on the housing part 3 to guide the clamping arms 8, 9. As a result, a consistently central arrangement can be ensured relative to the housing part 3 and to the breathing tube upon depth-adjustment. What is more, a certain spreading of the clamping arms 8, 9 upon moving in the longitudinal direction of the breathing tube 2 can also be brought about through catching on the housing part 3 by means of appropriate guide projections and/or guide slots.

As follows particularly from FIGS. 3 and 4, each clamping arm 8, 9 may have at its angled end 10, 11 a clamp roller 16, 17 which can be rotated about the longitudinal axis of the clamping arm 8, 9, the clamping area 12 being formed between the clamp rollers 16, 17. The spacing between the clamp rollers 16, 17 is dimensioned such that the nose is reliably sealed on the one hand but the clamping effect is not perceived as unpleasant by the test subject on the other hand. The pushing-up of the nose clip 4 on the nose is simplified here by the rotatable mounting of the clamp rollers 16, 17, the circumferential surfaces of the clamp rollers 16, 17 rolling down onto the outer nasal walls.

To ensure the best possible sealing of the nose, the clamp rollers 16, 17 can be cylindrical. For the same purpose, a provision can preferably be made for the clamp rollers 16, 17 to be constructed as an elastic molded body, particularly from foam. To adapt the position of the clamp rollers 16, 17 to the face and nose, they can be mounted such that they can be moved in the direction of the longitudinal axis X₂ of the clamping arms 8, 9. This becomes clear through a comparison of FIGS. 3 and 4.

For a rotatable mounting of the clamp rollers 16, 17, cylindrical roller bodies 18, 19 mounted such that they can be rotated about the longitudinal axis X₂ of the clamping arms 8, 9 can be provided on the angled ends 10, 11 of the clamping arms 8, 9, each clamp roller 16, 17 having a central opening for placement onto the roller body 18, 19. To adjust the position of the clamp rollers 16, 17 to the face and nose of the user 5, the clamp rollers 16, 17 can be held onto the roller bodies 18, 19 such that they can be moved in the axial direction. For this purpose, the roller body 18, 19 is longer than the clamp roller 16, 17. In addition, it is possible to use the clamp rollers 16, 17 in a disposable manner, in which case the clamp rollers 16, 17 can be removed from the roller bodies 18, 19 after one use and switched out for unused clamp rollers 16, 17. This ensures good hygiene during the determination of the output parameters.

Roller bodies 18, 19 are shown in a first embodiment in FIGS. 5 through 7. The roller bodies 18, 19 are secured by a retaining element 20, 21 against axial movement or sliding on the clamping arms 8, 9. In the depicted embodiment, a fixed shaft retaining ring is provided in a groove of the clamping arm 8, 9 as a retaining element 20, 21.

The roller body can be embodied in two parts and can have a cylindrical support section 22, 23 for the clamp rollers 16, 17 and an annular sealing member 24, 25. The sealing member 24, 25 can be a plastic washer.

To assemble the roller bodies 18, 19, the sealing member 24, 25 is first pushed or threaded onto the angled end 10, 11 of the respective clamping arm 8, 9. Then the retaining element 20, 21 is assembled. After that, the support section 22, 23 is pushed onto the clamping arm 8, 9 until it rests against the retaining element 20, 21. The support section 22, 23 has a central hole the diameter of which is slightly less than the diameter of a clamping arm 8, 9. This ensures the free rotatability of the roller body 18, 19.

In the embodiment depicted in FIGS. 5 through 7, the support section 22, 23 has on one end a radial land 26, 27 with an annular outer part 28, 29, so that a receiving area for the sealing member 24, 25 is formed. The radial land 26, 27 borders a circular recess 30, 31 which has a larger diameter than the shaft retaining ring. This makes it possible to adhere the sealing member 24, 25 to the support section 22, 23 at fewer points, thus securing the roller body 18, 19 against axial movement on the clamping arms 8, 9. One precondition for the free rotatability of the roller body 18, 19 is that it not be adhered to the clamping arm 8, 9.

To also ensure slight rotatability of the roller bodies 18, 19, a hollow space for the retaining element 20, 21 can be formed between the support section 22, 23 and the sealing member 24, 25 after adhesion, thus enabling slight compensatory movement of the roller body 18, 19 in the axial direction relative to the retaining element 20, 21. In FIG. 5, the roller bodies 18, 19 are shown in the disassembled state, while they are shown in the assembled state in FIG. 7.

An alternative embodiment of a roller body 18, 19 is shown in FIGS. 8 and 9. As in the embodiment described above, the roller body 18, 19, in turn, has a support section 22, 23 with a radial land 26, 27 and with an annular outer part 28, 29 on its end facing a sealing member 24, 25. However, in contrast to the embodiment depicted in FIGS. 5 through 7, a provision is made in the embodiment depicted in FIGS. 8 and 9 that the support section 22, 23 is connected in a non-positive and positive manner to the sealing member 24, 25 by means of a snap-on connection. For this purpose, axial slits 32 are provided in the annular outer part 28, 29, thus subdividing the annular outer part 28, 29 into several circle segments 33. When the sealing member 24, 25 is pressed into the receiving area formed between the radial land 26, 27 and the annular outer part 28, 29, the circle segments 33 can be reversibly bent outwardly against the material restorative forces, the snap forces to be overcome being minimal as a result of the slitted design.

The annular outer part 28, 29 has, when viewed in the axial direction, an inner bevel 34 and an outer bevel 35. This is shown particularly in FIG. 9. The outer bevel 35 has a lesser slope than the inner bevel 34, which results, for one thing, in less effort being required to depress the sealing member 24, 25 into the receiving area and, for another, in sufficient axial support of the roller body 18, 19 being ensured upon engagement of the sealing member 24, 25.

In the embodiment depicted in FIGS. 8 and 9 as well, a hollow space 36 is provided between the sealing member 24, 25 and the radial land 26, 27 in the assembled state of the roller body 18, 19 in order to ensure easy rotatability of the roller body 18, 19. The embodiment depicted in FIGS. 8 and 9 enables easy assembly of the roller bodies 18, 19. In addition, the sealing member 24, 25 can be separated again from the support section 22, 23 as needed for disassembling the roller bodies 18, 19.

The radial land 26, 27 on the support section 22, 23 acts as a stop for the clamp roller 16, 17. The range of motion of the clamp roller 16, 17 on the support section 22, 23 is therefore limited at the bottom.

The chin rest 6 shown in FIGS. 1 and 2 can also be mounted in a depth-adjustable manner in the axial direction of the breathing tube 2 and be fixable on the housing part 3 at various places. As a result, the position of the chin rest 6 can be adapted to the face of the user 5, thus ensuring a position that is comfortable for the user 5 during the determination of the output parameters. The clamping of the chin rest 6 on the housing part 3 can be commensurate with the described clamping of the nose clip 4 on the housing part 3.

The chin rest 6 can have a substantially U-shaped support bracket 37 with two support arms 38, 39, the two support arms 38, 39 being optionally bent in an L shape and connected to the housing part 3. The support bracket 37 can be embodied as a closed wire loop.

The two support arms 38, 39 are connected to each other and form a substantially U-shaped support area 40 to rest against the chin of the user 5, the two support arms 38, 39 being optionally arranged in the support area 40 at an angle of less than or equal to 90° to the central longitudinal axis X₁ of the breathing tube 2. The bend in the support arms 38, 39 can correspond to the bend of the free ends 10, 11 of the clamping arms 8, 9 of the nose clip 4. This renders the user unit 1 comfortable to wear.

As follows from FIG. 2 as well, the two support arms 38, 39 can be fixable to the housing part 3 by means of a screw joint using at least one clamping screw. The support arms 38, 39 can be tensioned here—commensurately with the clamping of the nose clip 4—between a screw head 41 of the clamping screw and the housing part 3 with a certain amount of spacing from the chin of the user 5.

It is not shown in detail that at least one guide projection and/or guide slot can be provided on the bottom side of the housing part 3 and/or on the screw head 41 to guide the support arms 38, 39.

In the following, other features and aspects of the invention are cited which describe possible embodiments in and of themselves or in any combination with each other and/or with the features cited in the patent claims and are therefore also essential to the invention. For instance, a provision can be made in the user unit according to the invention
that the free ends (10, 11) of the clamping arms (8, 9) are arranged at an angle of less than or equal to 90° to the central longitudinal axis (X₁) of the breathing tube (2), and/or
that the clamping bracket (7) can be fixed to the housing part (3) by means of a screw joint using at least one clamping screw, the clamping screw being optionally screwed between the clamping arms (8, 9) into the housing part (3) and the clamping bracket (7) being optionally tensioned between a screw head (15) of the clamping screw and the housing part (3), and/or
that at least one guide projection and/or guide slot is provided on the housing part (3) and/or on the screw head (15) to guide the clamping arms (8, 9), and/or
that the clamp roller (16, 17) is cylindrical, and/or
that the clamp roller (16, 17) is made as an elastic shaped body, particularly from foam, and/or
that the clamp roller (16, 17) is mounted such that it can be moved in the direction of the longitudinal axis (X₂) of the clamping arm (8, 9), and/or
that the roller body (18, 19) is longer than the clamp roller (16, 17), and/or
that at least one retaining element (20, 21) is provided to secure the roller body (18, 19) against axial movement on the clamping arm (8, 9), preferably a shaft retaining ring fixed in a groove of the clamping arm (8, 9), and/or
that the roller body (18, 19) is embodied in two parts and has a cylindrical support section (22, 23) and a sealing member (24, 25), wherein the support section (22, 23) is rotatably mounted on the clamping arm (8, 9) and rests against the retaining element (20, 21), wherein the sealing member (24, 25) is connected to the support section (22, 23) in a non-positive and/or positive and/or adhesive manner and wherein, preferably, a hollow space is formed between the support section (22, 23) and the sealing member (24, 25) for the retaining element (20, 21), and/or
that the support section (22, 23) has a radial land (26, 27) as a stop for the clamp roller (16, 17) at its bottom end, and/or
that the two support arms (38, 39) are connected to each other and form a substantially U-shaped support area (40) to rest against the chin of the user (5), the support area (40) being arranged at an angle of less than or equal to 90° to the central longitudinal axis (X₁) of the breathing tube (2), and/or
that the two support arms (38, 39) can be fixed to the housing part (3) by means of a screw joint using at least one clamping screw, the support arms (38, 39) being optionally tensioned between a screw head (41) of the clamping screw and the housing part (3).

1. A user unit for determining output parameters from respiratory gas analyses, particularly for use in spiroergometry devices, with a preferably replaceable breathing tube and a housing part, wherein the breathing tube, also preferably, can be inserted into the housing part in order to put the user unit into a measuring mode, characterized in that a nose clip connected to the housing part (3) and designed to seal the nose of a user during the determination of the output parameters is provided on the top side of the housing part and a chin rest connected to the housing part and designed to rest against the chin of a user during the determination of the output parameters is provided on the bottom side of the housing part, wherein the nose clip is mounted on the housing part such that it can be depth-adjusted in the axial direction of the breathing tube and fixed at various points on the housing part and wherein the chin rest is mounted on the housing part such that it can be depth-adjusted in the axial direction of the breathing tube and fixed at various points on the housing part.

2. The user unit as set forth in claim 1, wherein after the insertion of the breathing tube into a test subject's mouth, the housing part can be held on the test subject's head merely by means of the nose clip and the breathing tube and Supported on the test subject's chin by means of the chin rest.

3. The user unit as set forth in claim 1, wherein the nose clip has a substantially U-shaped clamping bracket with two clamping arms, wherein the two clamping arms are angled in an L shape at the free ends and wherein a clamping area for insertion of the nose that is open to the side of a mouthpiece of the breathing tube is formed between the angled ends.

4. The user unit as set forth in claim 1, wherein each clamping arm has at its angled end a clamp roller that can be rotated about the longitudinal axis (X₂) of the clamping arm, the clamping area being formed between the clamp rollers.

5. The user unit as set forth in claim 1, wherein a cylindrical roller body that is mounted such that it can rotate about the longitudinal axis (X₂) of the clamping arm is provided for the clamp roller on the angled end of the clamping arm, the clamp roller having a central opening for placement onto the roller body and, preferably, being movable on the roller body in the axial direction.

6. (canceled)

7. (canceled)

8. (canceled)

9. The user unit as set forth in claim 1, wherein at least one guide projection and/or a guide slot is provided on the housing part and/or on the screw head to guide the support arms.

10. (canceled)

11. The user unit as set forth in claim 1, wherein the chin rest has a substantially U-shaped bracket with two support arms, the two support arms being bent in an L shape and connected to the housing part.
